(19) United States
(12) Patent Application Publication
(10) Pub. No.: US 2007/0181126 A1
(43) Pub. Date: Aug. 9, 2007

(54) Method and apparatus for ventilating a patient with a breathing gas mixture formed from nitric oxide, air, and oxygen

(76) Inventors: Craig R. Tolmie, Stoughton, WI (US); Thomas S. Kohlmann, McFarland, WI (US); Robert Q. Tham, Middleton, WI (US); Karl N. Knauf, Madison, WI (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/349,753

(22) Filed: Feb. 8, 2006

(51) Int. Cl. A61M 16/00; A62B 7/00

(52) U.S. Cl. 128/204.21; 128/204.18

(57) ABSTRACT

A method and apparatus for supplying a breathing gas mixture to a patient in which a desired concentration of oxygen is maintained when nitric oxide (NO) is provided in the breathing gases. A clinician establishes at a ventilator, ventilation parameters for the patient, the inspired oxygen concentration, and the inspired NO dosage. From these quantities, a breathing gas mixture flow rate is determined. An instantaneous flow rate for an NO containing gas is determined, based on the concentration of nitric oxide in the supply gas and the instantaneous breathing gas mixture flow rate. An instantaneous flow rate for the supply of a balance gas, such as air, is determined using the breathing gas mixture flow rate, the instantaneous NO containing gas flow rate, and the inspired oxygen concentration established by the clinician. Finally, the instantaneous oxygen flow rate is determined as the difference between the inspiratory breathing gas flow rate and the instantaneous flow rates for the NO containing gas and the balance gas. A breathing gas mixture is thereafter provided to the patient at an instantaneous flow rate comprising the sum of the NO containing gas, balance gas, and oxygen flow rates. Actual gas flows are sensed by gas flow sensors and used to render the gas flow rates and the concentrations of NO and oxygen more accurate.

### FIELD OF THE INVENTION

The present invention relates to a method and apparatus for administering inhaled nitric oxide (NO) to a patient or other subject while ensuring that a desired concentration of oxygen is also delivered to the patient. The invention may be used to ensure that a minimum concentration of oxygen is delivered to the patient so that the breathing gases provided to the patient and containing the nitric oxide do not become hypoxic.

### BACKGROUND OF THE INVENTION

Nitric oxide is a gas that, when inhaled, acts to dilate blood vessels in the lungs, improving oxygenation of the blood and reducing pulmonary hypertension. For this purpose, the nitric oxide is provided in the inspiratory breathing gases for the patient. The dosages of nitric oxide are small, typically 150 parts per million (ppm) or less.

Commercially available supplies of nitric oxide comprise pressurized tanks containing nitric oxide in an inert diluent gas, such as nitrogen. The nitric oxide is typically present in a concentration of 800 parts per million. While this facilitates administration of the nitric oxide, since valves or other control apparatus can work with larger volumes of gas, it also means that a larger volume of gas, that is mainly inert, is added to the breathing gases for the patient.

For patients breathing with the aid of a mechanical ventilator, the patient is supplied with breathing gases from the ventilator by a breathing circuit. The ventilator is connected to a source of oxygen and a source of a balance gas, typically air. The supply of NO may also be connected to the ventilator, but is more commonly connected to the breathing circuit to provide the NO in the breathing gases prior to inspiration by the patient.

It will be appreciated that the NO delivery will increase the volume of gas in the breathing circuit. For example, the delivery of 80 ppm NO from an 800 ppm NO supply will add 10% more gas to that delivered by the ventilator. If the concentration of oxygen delivered by the ventilator is 50% on a volume basis, following provision of the 80 ppm NO dose, and the resulting 10% increase in gas volume, the concentration of oxygen inspired by the patient will be only 45% on a volume basis. This dilution of inspired oxygen as a result of NO provision may not be fully understood by a clinician setting the operating parameters of the ventilator, such as the volume and/or pressure characteristics of gas delivery by the ventilator, as well as the composition of the breathing gases. It is potentially dangerous to the patient since at lower oxygen concentrations and higher NO dosages it could lead to the delivery of hypoxic breathing gases to the patient, i.e. breathing gases with an insufficient amount of oxygen for the physiological functioning of the patient. Also, the provision of the NO containing gas causes the tidal volume delivered to the patient to be greater than that set on the ventilator and possibly higher than that desired to be delivered to, the patient and may cause problems in the regulation of the ventilator during volume controlled ventilation.

A nitric oxide delivery system may be included in a ventilation system as a generally independent apparatus that is used in conjunction with an existing ventilation system, as is described in Bathe, et al U.S. Pat. No. 5,558,083 and Stenzler U.S. Pat. No. 6,581,599. These systems, external to the ventilation system, provide an efficient way to add NO delivery capability to existing ventilator products and are usable with a variety of different ventilation products from a variety of different manufacturers. However, as noted above, problems may attend externally adding additional NO containing gases to the breathing circuit in the absence of proper communication between the NO delivery system and the mechanical ventilator. Also, as the NO delivery system is not integral with the ventilator, the NO delivery system requires a flow sensor to measure the other components of the breathing gases. For ease of use, this flow sensor and the NO injection device are often combined with a single component placed in the breathing circuit. For certain types of flow sensors, such as hot wire anemometers, the flow sensor—injection component is preferably placed upstream of a breathing gas humidifier in the breathing circuit. However, this results in a period of transit time in the breathing circuit in which the NO gas is in contact with the oxygen in the breathing gases and can form toxic NO₂ gas prior to delivery to the patient.

In the expiratory limb of the breathing circuit, a measurement taken by a flow sensor is used to monitor the mechanical ventilation of the patient. This monitoring includes the detection of spontaneous breathing attempts by the patient in mechanically assisted ventilation. To detect and assist spontaneous breathing, a constant bias flow of breathing gas is provided through the breathing circuit to reduce the airway resistance and aid the spontaneous breathing. The expiratory limb flow sensor detects changes in this bias flow rate as indicative of a patient's attempt to spontaneously breathe at which point the ventilator refrains from providing a breath or provides such breathing assistance as is needed. That is, a spontaneous inhalation by the patient will reduce the bias flow in the expiratory limb which is detected by the flow sensor as a spontaneous breathing attempt. The addition of extra NO gas to the patient breathing circuit external to the ventilator creates a quantity of additional gas in the patient's breathing circuit that the ventilator is unaware of and may affect the ability of the ventilator to detect spontaneous breathing by the patient.

Additionally, a sample of the breathing gases may be taken from the breathing circuit before they are delivered to the patient. This is for analysis to determine the content of the gases delivered to the patient, as disclosed in Bathe, et al. This gas sample removes a portion of the total gas supplied to the patient thus reducing the flow rate seen in the expiratory limb of the patient breathing circuit. The changes in expiratory limb conditions resulting from gas sample removal may be seen by the ventilator as an attempt by the patient to spontaneously breath; therefore the ventilator will refrain from mechanically assisting the patient's ventilation. While an appropriate trigger level is provided in the ventilator to prevent or minimize such occurrences, the addition of the NO containing gas may hinder the operation of this trigger.

The same situation also exists with respect to the detection of leaks in the breathing circuit. Leaks, such as those occurring at a face mask for the patient, are commonly sensed by detecting reduced gas flows in the expiratory limb of the breathing circuit. The addition of the NO containing gas may alter the ability of the ventilator to detect leaks in this manner.

Still further, it is often desired to measure the amount of oxygen consumed by a patient. This is the difference between the amount of oxygen inspired and the amount of oxygen expired, commonly termed VO₂. While the amount of oxygen inspired is known from the operation of the ventilator, measuring the amount of expired oxygen requires measuring the expiratory gas flow, which flow may be altered by the injection of NO containing gas. Such alterations may not be taken into account when determining VO₂.

### SUMMARY OF THE INVENTION

In the present invention, a central processing unit in the ventilator is connected by a data bus to a central processing unit in the NO delivery device so that data needed for, and resulting from, the administration of NO maybe used in a coordinated fashion. The accuracy by which concentrations of O₂ and NO are administered to the patient is thereby enhanced.

More particularly, an embodiment of the present invention provides a method and apparatus for supplying a breathing gas mixture to a patient in which a desired concentration of oxygen is maintained when NO is provided in the breathing gases, thereby to insure that hypoxic breathing gases are not delivered to a patient. Spontaneous breathing by the patient and leaks in the patient limb of the breathing circuit can be detected using information representative of the amount of NO added to the breathing circuit as well as the amount of gas removed from the breathing circuit by a gas analyzer for analysis purposes.

To carry out the invention, a clinician establishes at a ventilator, ventilation parameters for the patient, the inspired oxygen concentration, and the inspired NO dosage. From these quantities, a breathing gas mixture inspiratory flow rate for the ventilator is determined. An instantaneous flow rate for the NO containing gas is determined, based on the concentration of nitric oxide in the supply gas and the instantaneous breathing gas mixture flow rate. An instantaneous flow rate for the supply of a balance gas is determined using the breathing gas mixture inspiratory flow rate, the instantaneous NO flow rate, and the inspired oxygen concentration established by the clinician. The balance gas will typically be air but other gases or mixtures thereof may be used. Finally, the instantaneous oxygen flow rate is determined as the difference between the inspiratory breathing gas flow rate and the instantaneous flow rates for the NO containing gas and the balance gas. A breathing gas mixture is thereafter provided to the patient at an instantaneous flow rate comprising the sum of the NO containing gas, balance gas and oxygen flow rates. Actual gas flows are sensed by gas flow sensors and used to render the gas flow rates and concentrations of NO and oxygen more accurate.

### BRIEF DESCRIPTION OF THE DRAWING

The invention will be more fully appreciated from the following detailed description, taken in conjunction with the figures in which:

FIG. 1 shows apparatus in accordance with the present invention; and

FIG. 2 is a flow chart illustrating the steps of the method of the present invention.

### DETAILED DESCRIPTION

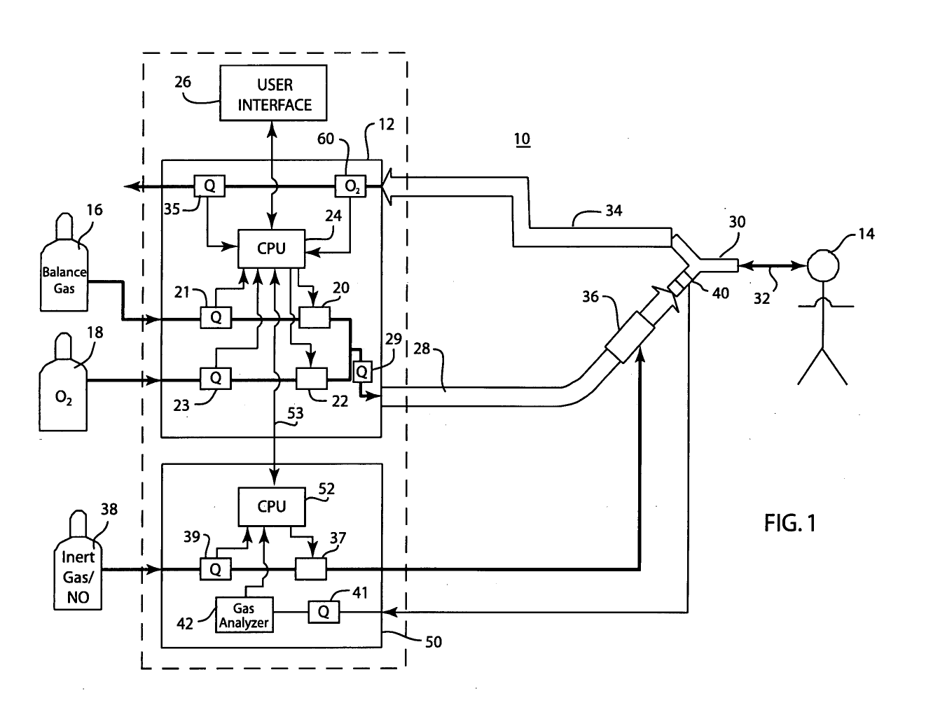

FIG. 1 shows apparatus 10 of an embodiment of the present invention incorporating mechanical ventilator 12 for supplying breathing gases to a patient 14.

Ventilator 12 receives a balance gas from a source 16, which may comprise a gas such as air, nitrogen or helium. Ventilator 12 also receives oxygen from a source 18. While oxygen source 18 is shown as a pressurized tank in FIG. 1, it will be appreciated that other sources may be used, such as an oxygen supply manifold commonly found in a hospital setting. The flow of balance gas is measured by flow sensor 21 and controlled by valve 20 in, ventilator 12 and the flow of oxygen is similarly measured by flow sensor 23 and controlled by valve 22. The operation of valves 20 and 22 is established by a control device such as central processing unit 24. A user interface 26 allows the clinician to establish the operating parameters of ventilator 12 for ventilating patient 14, as well as the desired concentration for oxygen and dosage amount of NO. The user interface 26 may also include a display for monitoring the operation of ventilator 12.

Ventilator 12 supplies breathing gases comprising the mixture of balance gas and oxygen, as controlled by valves 20 and 22, respectively, to inspiratory limb 28 of a breathing circuit. The flow of the mixed gases may be sensed by flow sensor 29. Inspiratory limb 28 is connected to Y-connector 30 and to patient limb 32 that provides inspiratory breathing gases to the patient 14 and receives expiratory breathing gases from the patient. Breathing gases expired by the patient 14 are discharged through expiratory limb 34 of the breathing circuit. The breathing gases expired by the patient are measured by flow sensor 35 in the path for the expired breathing gases. After the exhaled breathing gases are measured by flow sensor 35, the exhaled gases may be provided to a gas scavenger system or vented directly to ambient air. Appropriate check valves (not shown) are provided in the breathing circuit to cause the breathing gases to flow in the above described manner. Ventilator 12 typically provides a small, continuous bias gas flow through the breathing circuit

A NO delivery apparatus 50 provides NO to the breathing gases for patient 14. While NO may be provided through ventilator 12 in the same manner as the balance gas and oxygen, because NO reacts with oxygen to form nitrogen dioxide (NO₂), a toxic compound, it is deemed preferable to provide the NO into the breathing gases of the patient as close to the patient as possible. However, in contrast to the more stand-alone approach of systems such as that shown in Bathe et al. '083, in the present invention, NO delivery apparatus 50 maybe integral with the ventilator with communication being present between the NO delivery apparatus and ventilator 12. Since the measurement of the gas flow supplied by the ventilator to the breathing circuit is carried out by flow sensor 29, a separate flow sensor is not necessary in the breathing circuit and NO injection device 36 may be placed closer to the patient and downstream of a humidifier (not shown). This reduces the time that the NO and O₂ are in contact before being inspired, and thus reduces the formation of toxic NO₂ gas.

As shown in FIG. 1, NO injection device 36 provides NO to the breathing circuit in inspiratory limb 28 at a location proximal to Y connector 30. NO injection device 36 is coupled through NO delivery apparatus 50 to a supply of nitric oxide 38 that, as described above, is typically NO in a diluent inert gas, such as nitrogen or helium, in a pressurized tank. NO delivery apparatus 50 also includes central processing unit 52 for controlling the operation of valve 37 supplying gas to NO injection device 36. NO delivery apparatus 50 also includes flow sensor 39.

CPU 52 of NO delivery apparatus 50 communicates with CPU 24 of the ventilator 12 via data bus 53. By this communication, despite NO delivery apparatus 50 being a separate component, the CPU 24 of ventilator 12 views NO delivery apparatus 50 in a coordinated and unity manner. The communication provides ability for the enhanced breathing gas delivery and control of the present invention.

A gas sampling port 40 is provided in the inspiratory limb of Y-connector 30 for withdrawing a sample of the breathing gases supplied to patient 14. The sample is provided through flow sensor 41 to gas analyzer 42 which is coupled to central processing unit 52.

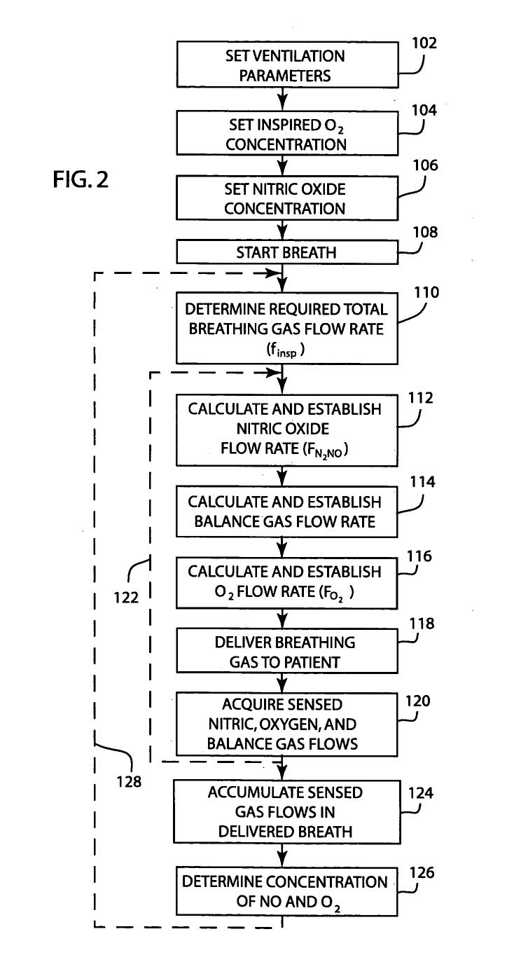

The flow chart of FIG. 2 shows a method of the present invention.

Using user interface 26, a clinician sets the ventilation parameters for patient 14, such as breath rate, inspiratory/expiratory (I:E) ratio, tidal volume, minute volume, breathing gas pressures, and the like. This is carried out in step 102. The parameters set by the clinician also include the concentration of oxygen to be inspired by the patient as well as the dosage or concentration of NO to be delivered to the patient. While this would ordinarily be included as part of step 102, the setting of these parameters is shown as separate steps 104 and 106 in FIG. 2 to facilitate an understanding of the present invention.

Thereafter, ventilator 12 is operated to commence the provision of breathing gases to patient 14 in an inspiratory phase of the respiratory cycles at step 108.

Central processing unit 24 then computes a required instantaneous flow rate for the breathing gas mixture, (fᵢₙₛₚ) in step 110 based on ventilator settings for the patient. It will be appreciated that as the supply of breathing gases to the patient proceeds, the instantaneous flow rate will vary during the course of an inspiration by the patient, being high at the beginning of inspiration when the lungs are empty, and lessening as the patient's lungs fill. Typical instantaneous flow rates will vary from 10 to 2 liters/min.

The instantaneous delivery rate (F_{N2NO}) for the NO containing gas from supply 38 via NO injection device 36 is determined in step 112 in accordance with the following formula.

$$Instantaneous NO delivery rate ( {F}_{{N}_{2} NO} ) = \frac{{F}_{ino\_set} \times {f}_{insp}}{{F}_{NO\_supple}} \qquad ( 1 )$$

It will be appreciated that the instantaneous NO containing gas delivery rate is determined by ratioing the NO dosage to the nitric oxide supply concentration and applying the ratio to the instantaneous breathing gas mixture flow rate (fᵢₙₛₚ). For example, if NO is to be supplied to the patient at a dosage of 80 ppm from a nitric oxide supply 38 in which the concentration is 800 ppm, the ratio of these two quantities is 0.1. If the instantaneous inspiratory flow rate for the breathing gas mixture is 10 liters/min, the flow rate for the NO containing gas is 1 liter/min. This determination may be carried in either CPU 24 or CPU 52 and communicated via bus 53. It is preferably carried out in CPU 24 of ventilator 12 in as much as this CPU contains the mixture control algorithms, resulting in a high level of NO delivery accuracy. The instantaneous NO delivery flow rate so determined is delivered through control valve 37 to NO injection device 36 to provide the desired dosage of NO in the breathing gas mixture to patient 14 from supply 38.

As a result of the determinations made at steps 110 and 112, the instantaneous inspiratory breathing gas mixture flow rate (fᵢₙₛₚ) commanded by ventilator 12 and the instantaneous delivery rate for the NO containing gas (F_{N2NO}) are both known. The difference between these two flow rates is the flow rate for oxygen containing breathing gases. In the case of the present exemplary embodiment, these gases comprise both air and oxygen from supply 18. A balance gas comprising air is 21% oxygen and 79% non-oxygen and the gas from oxygen source 18 is 100% oxygen. Air and oxygen are provided in the relative amounts necessary to establish the oxygen concentration in the breathing gas mixture at that set by the clinician in step 104.

To commence the determination of the foregoing amounts, the instantaneous air flow rate is next determined as

$$Instantaneous air flow rate ( {F}_{air} ) = \frac{( ( 1 - {F}_{i O 2} ) \times {f}_{insp} ) - {F}_{N 2 NO}}{0.79} \qquad ( 2 )$$

The quantity F_{iO2} is the inspired oxygen concentration selected by the clinician in step 104 and fᵢₙₛₚ is the instantaneous flow rate for the breathing gas mixture. The quantity 0.79 is the fraction of air that is not oxygen. The result of the calculation step 114 is a flow rate for air, 21% of which will be oxygen.

Finally, the instantaneous oxygen flow rate is determined at a level such that the amount of oxygen supplied by this flow when taken with the amount of oxygen contained in the air flow will ensure that the inspired oxygen concentration selected by the clinician will be provided to patient 14 in the breathing gas mixture. This flow rate is determined in step 116, as
Instantaneous O₂ flow rate(F_{O2})=fᵢₙₛₚ−F_{N2NO}−Fₐᵢᵣ (3)

It will be appreciated from Equation 3 that the instantaneous oxygen flow rate (F_{O2}) is the difference between the instantaneous flow rate (fᵢₙₛₚ) commanded by ventilator 12 and the sum of the instantaneous NO containing gas flow rate (F_{N2NO}) and the instantaneous air flow rate (Fₐᵢᵣ).

CPU 24 commands valves 37, 20, and 22 to provide the determined instantaneous flow rates for the NO containing gas, air, and oxygen, respectively, in step 118 to provide breathing gases to patient 14 at the total inspiratory flow rate (fᵢₙₛₚ) determined in step 110.

Under the control of CPU 24 in ventilator 12, steps 110 through 118 are periodically repeated during the inspiratory phases of the respiratory cycles of the patient as the instantaneous breathing gas mixture flow rate commanded by the ventilator changes. For example, the steps may be repeated every 2 milliseconds to determine new instantaneous total flow rates and instantaneous flow rates for the NO containing gas, air, and oxygen.

As the operation of ventilator 12 occurs, flow sensors 21, 23, and 39 sense the actual flow rates for the gases. See step 120. This data is acquired and provided to one or both of the CPUs in ventilator 12 and/or NO delivery apparatus 50 at step 120. This acquired data is then used in subsequent calculation of the flows of NO containing gas, balance gas, and oxygen at steps 112, 114, and 116, as shown by line 122 to improve the accuracy by which the flow rates are established as the delivery of breathing gas to patient 14 in a given breath proceeds.

The flows of, particularly, the NO containing gas and the oxygen gas are acquired by the flow sensors and accumulated or summed over the course of a breath in step 124. Knowing the flows of these gases and the concentrations in the gas sources 38 and 18, respectively, the actual concentration of NO and oxygen delivered to patient 14 in a given breath can be determined in step 126. The concentration so determined are then compared to the NO and oxygen concentrations set in steps 104 and 106, as indicated by line 128 and any differences between set and actual concentrations used in steps 112 et seq. performed in a subsequent breath to improve the accuracy with which NO and oxygen are delivered in the subsequent breath.

The delivery of the set oxygen concentration established by the clinician to patient 14 and the avoidance of a hypoxic breathing gas mixture is assured, as follows. As noted above, this assurance is most particularly needed in cases in which the NO dosage to the patient is increased so that a greater portion of the breathing gas mixture is the NO containing gas. Thus, if the amount of NO to be delivered to patient 14 (Fᵢₙₒ_{_{—}}ₛₑₜ) is increased, the instantaneous NO delivery rate (F_{N2NO}) will be increased, in accordance with Equation 1, assuming the concentration of NO in supply 38 remains the same. From an inspection of Equation 2, it will be appreciated that an increase in the instantaneous NO delivery rate (F_{N2NO}), will decrease the numerator in Equation 2, inasmuch as the instantaneous nitric oxide delivery rate (F_{N2NO}) is subtracted from the other quantity in the numerator of that equation. This in turn will decrease the instantaneous air flow rate F_{Air} as determined by Equation 2. In Equation 3, while the smaller instantaneous NO delivery rate (F_{N2NO}) is increased, the larger instantaneous air flow rate (F_{Air}), is decreased. These quantities when subtracted from the instantaneous inspiratory breathing gas mixture flow rate (fᵢₙₛₚ), results in an increase in the instantaneous oxygen flow rate (F_{O2}) determined by Equation 3 that ensures that the inspired oxygen concentration selected by the clinician continues to be delivered to patient 14.

When ventilator 12 is assisting spontaneous breathing efforts by patient 14, the system uses flow sensor 35 in the expiratory limb 34 to detect a reduction in the bias flow established by ventilator 12 in expiratory limb 34 when the patient attempts to inhale. By providing a bias flow, the work of the patient's respiration is minimized because the patient can use the existing continuous gas flow rather than being required to initiate a flow of breathing gases through the breathing circuit. However, the addition of NO containing gas in the breathing circuit increases the bias flow so that the ventilator may not detect an attempt by the patient to inhale.

To avoid this, the bias flow may be compensated for the amount of NO containing gas that has been added to the breathing circuit so as to maintain the bias flow at a desired, constant, preferably low, level for the detection of spontaneous breathing. Flow sensor 39 may be used to determine the amount of NO containing gas that is added to the breathing circuit. The bias flow may be decreased to compensate for the additional NO containing gas that is supplied to the breathing circuit. Alternatively, the trigger level in the ventilator may be adjusted by a signal from NO delivery apparatus 50 in data bus 53 to compensate for the addition of the NO containing gas to the bias flow.

Analogous, but opposite, steps may be taken to compensate the bias flow for amounts of gas that are removed from the breathing circuit for use by gas analyzer 42. Flow sensor 41 in NO delivery apparatus 50 may be used to determine the amounts of gas removed at gas sampling port 40.

Additionally, the flow sensor 35 in expiratory limb 34 can be used to detect the presence of leaks in the breathing circuit, especially in the interface between the patient limb 32 and patient 14. This interface may be a mask or endotracheal tube or other similar device. With the compensation for expiratory gas flow changes or to the trigger level provided using the data from NO delivery apparatus 50, leaks can be properly detected as by the reduction in the bias gas flow resulting from the leak.

Also, the calculation of the data quantity VO₂, which is the amount of oxygen consumed by patient 14, i.e. the difference between the amount of oxygen inspired and the amount of oxygen expired by patient 14 can be improved through use of the present invention.

The possibility exists for VO₂ to be computed inaccurately due to the addition of the NO containing gas external to the ventilator 12 as the flow sensor 35 may read a greater flow of gas in the expiratory limb 34 than was provided to the inspiratory limb 28 by the ventilator 12 and thus an incorrect amount of expired oxygen. To avoid this, CPU 52 of NO delivery apparatus 50 may provide compensation data to CPU 24 of ventilator 12 that is indicative of the additional NO containing gas that is introduced to the breathing circuit external to the ventilator to provide a correct value for the expiratory gas flow rate. An oxygen sensor 60 may be inserted in the path for the expired breathing gases in the expiratory limb or in ventilator 12 to sense the level of oxygen in the expired breathing gases for use with expiratory gas flow rate to accurately determine the amount of expired oxygen. The amount of oxygen inspired will be known from the operation of ventilator 12.

Various alternatives and embodiments are contemplated as being within the scope of the following claims particularly pointing out and distinctly claiming the subject matter regarded as the invention.

What is claimed is:

1. A method for administering inhaled nitric oxide to a subject breathing with the aid of a mechanical ventilator, a gas mixture for the subject being formed from supplies of oxygen, a balance gas, and a gas containing nitric oxide, said method comprising the steps of:
(a) setting ventilation parameters, inspired oxygen concentration, and inspired nitric oxide concentration for the subject;
(b) determining from the settings of step (a) a total gas mixture flow rate (fᵢₙₛₚ) for the subject;
(c) determining a flow rate for the nitric oxide containing gas (F_{N2NO}) based on the gas mixture flow rate (fᵢₙₛₚ);
(d) determining a flow rate for the balance gas (F_{Air}) from the total gas mixture flow rate, the nitric oxide containing gas flow rate, and the inspired oxygen concentration established in step (a);
(e) determining the oxygen flow rate (F_{O2}) as the difference between the total gas mixture flow rate (Fᵢₙₛₚ) and the inspiratory flow rate for the nitric oxide gas mixture (F_{N2NO}) and balance gas (F_{Air}); and
(f) providing the breathing gas mixture to the subject at a flow rate comprising the sum of the flow rate for the nitric oxide containing gas, the balance gas flow rate, and the oxygen flow rate.

2. The method according to claim 1 wherein the flow rates are instantaneous flow rates.

3. The method according to claim 1 further defined as determining the concentration of nitric oxide in the nitric oxide containing gas and using the determination of nitric oxide concentration in the nitric oxide containing gas in determining the flow rate for the nitric oxide containing gas.

4. The method according to claim 1 further defined as including the step of sensing the flow rate of at least one gas provided in the breathing gas mixture to the subject during a breath and using the sensed flow rate in determining the flow rates for the gases of the gas mixture in the breath.

5. The method according to claim 2 further defined as including the step of sensing the flow rate of at least one gas provided in the breathing gas mixture to the subject during a breath and using the sensed flow rate in determining the flow rates for the gases of the gas mixture in the breath.

6. The method according to claim 1 further defined as including the step of sensing flow rates of a plurality of gases provided in the breathing gas mixture to the subject during a breath and using the sensed flow rates in determining the flow rates for the gases of the gas mixture in the breath.

7. The method according to claim 2 further defined as including the step of sensing flow rates of a plurality of gases provided in the breathing gas mixture to the subject during a breath and using the sensed flow rates in determining the flow rates for the gases of the gas mixture in the breath.

8. The method according to claim 1 further defined as including the step of sensing the flow rate of at least one gas provided in the breathing gas mixture to the subject in the course of a breath and using the sensed flow rate in determining the flow rates for the gases of the gas mixture provided in a subsequent breath.

9. The method according to claim 2 further defined as including the step of sensing the flow rate of at least one gas provided in the breathing gas mixture to the subject in the course of a breath and using the sensed flow rate in determining the flow rates for the gases of the gas mixture provided in a subsequent breath.

10. The method according to claim 1 further defined as including the step of sensing flow rates of a plurality of gases provided in the breathing gas mixture to the subject in the course of a breath and using the sensed flow rates in determining the flow rates for the gases of the gas mixture provided in a subsequent breath.

11. The method according to claim 2 further defined as including the step of sensing flow rates of a plurality of gases provided in the breathing gas mixture to the subject in the course of a breath and using the sensed flow rates in determining the flow rates for the gases of the gas mixture provided in a subsequent breath.

12. The method according to claim 1 wherein the balance gas and oxygen are supplied to the breathing gas mixture by the ventilator.

13. The method according to claim 12 wherein the nitric oxide containing gas is supplied to the breathing gas mixture by a nitric oxide delivery apparatus.

14. The method according to claim 13 further defined as controlling the operation of the ventilator and the nitric oxide delivery apparatus in a coordinated fashion to provide the breathing gas mixture to the subject.

15. The method according to claim 13 further defined as including the step of determining amounts of nitric oxide containing gas supplied in the breathing gas mixture provided to the subject for use in the detection of conditions altering breathing gas flows in a breathing circuit for the subject.

16. The method according to claim 13 further defined as including the step of determining amounts of breathing gas mixture removed from a breathing circuit for the subject for use in the detection of conditions altering breathing gas flows in the breathing circuit.

17. The method according to claim 1 wherein the balance gas is air.

18. Apparatus for administering inhaled nitric oxide (NO) to a subject, a breathing gas mixture for the subject being formed from supplies of oxygen, a balance gas, and a gas containing NO, said apparatus comprising:
a ventilator couplable to the supplies of oxygen and the balance gas and having control means for controlling the flow of air oxygen and the balance gas;
an NO delivery apparatus couplable to the supply of NO containing gas and having control means for controlling the flow of NO containing gas, the control means of said ventilator and the control means of said NO delivery apparatus being in data communication;
means for setting ventilation parameters, inspired oxygen concentration, and inspired NO concentration for the subject;
means for determining a total gas mixture flow rate (fᵢₙₛₚ) for the subject, a flow rate for the NO containing gas (F_{N2NO}), a flow rate for the balance gas (F_{Air}), and an oxygen flow rate (F_{O2}); and
said determining means causing said control means of said ventilator and NO delivery apparatus to provide a breathing gas mixture to the subject at a flow rate comprising the sum of the flow rate for the NO containing gas, the balance gas flow rate, and the oxygen flow rate.

19. The apparatus according to claim 18 wherein said determining means determines instantaneous flow rates.

20. The apparatus according to claim 18 further including means for sensing the flow rate of at least one gas provided in the breathing gas mixture to the subject during a breath and for using the sensed flow rate in determining the flow rates for the gases of the gas mixture in the breath.

21. The apparatus according to claim 19 further including means for sensing the flow rate of at least one gas provided in the breathing gas mixture to the subject during a breath and for using the sensed flow rate in determining the flow rates for the gases of the gas mixture in the breath.

22. The apparatus according to claim 18 further including means for sensing the flow rate of at least one gas provided in the breathing gas mixture to the subject in the course of a breath and for using the sensed flow rate in determining the flow rates for the gases of the gas mixture provided in a subsequent breath.

23. The apparatus according to claim 19 further defined as including the step of sensing flow rate of at least one gas provided in the breathing gas mixture to the subject in the course of a breath and using the sensed flow rate in determining the flow rates for the gases of the gas mixture provided in a subsequent breath.

24. The apparatus according to claim 18 further including means for determining amounts of nitric oxide containing gas supplied in the breathing gas mixture provided to the subject for use in the detection of conditions altering breathing gas flows in a breathing circuit for the subject.

25. The apparatus according to claim 18 further including means for determining amounts of breathing gas mixture removed from a breathing circuit for the subject for use in the detection of conditions altering breathing gas flows in the breathing circuit.

26. The apparatus according to claim 18 wherein the balance gas is air.
